(19) United States
(12) Patent Application Publication
(10) Pub. No.: US 2015/0182722 A1
(43) Pub. Date: Jul. 2, 2015

(54) INFANT CARE STATION HUMIDIFICATION MANAGEMENT SYSTEM

(71) Applicant: General Electric Company, Schenectady, NY (US)

(72) Inventors: Jacob Elliott Lesch, Washington, DC (US); Lawrence Guy Ten Eyck, Ellicott City, MD (US)

(73) Assignee: General Electric Company, Schenectady, NY (US)

(21) Appl. No.: 14/145,602

(22) Filed: Dec. 31, 2013

(51) Int. Cl. A61M 16/16; A61G 11/00

(52) U.S. Cl. CPC A61M 16/161; A61G 11/00; A61M 2205/18; A61G 2203/46; A61M 2240/00; A61G 2203/10; A61G 2210/30

(57) ABSTRACT

An infant care station humidification management system comprises an output device and a controller to generate control signals based upon a determined rainout condition. The output device provides output in response to the control signals.

### BACKGROUND

Humidity is sometimes added to a neonatal microenvironment of an infant care station or incubator to provide for a neutral thermal environment and enhance skin development. In certain circumstances, the added humidity within the infant care station may become condensation on the wall of the infant care station and the condensation may drip into the infant care station, a condition known as rainout. Such rainout may pose an infection hazard to the neonatal or infant.

### BRIEF DESCRIPTION OF THE DRAWINGS

FIG. 1 is a schematic illustration of an example infant care humidification management system.

FIG. 2 is a flow diagram of an example method that may be carried out by the system of FIG. 1.

FIG. 3 is a schematic illustration of another example infant care humidification management system.

FIG. 4 is a flow diagram of a first example method that may be carried out by the system of FIG. 3.

FIG. 5 is a flow diagram of a second example method that may be carried out by the system of FIG. 3.

FIG. 6 is a flow diagram of a third example method that may be carried out by the system of FIG. 3.

FIG. 7 is a perspective view of an example infant care station including the infant care humidification management system of FIG. 3.

FIG. 8 is a side view of the infant care station of FIG. 7.

FIG. 9 is a front view of the infant care station of FIG. 7.

### DETAILED DESCRIPTION OF THE EXAMPLE EMBODIMENTS

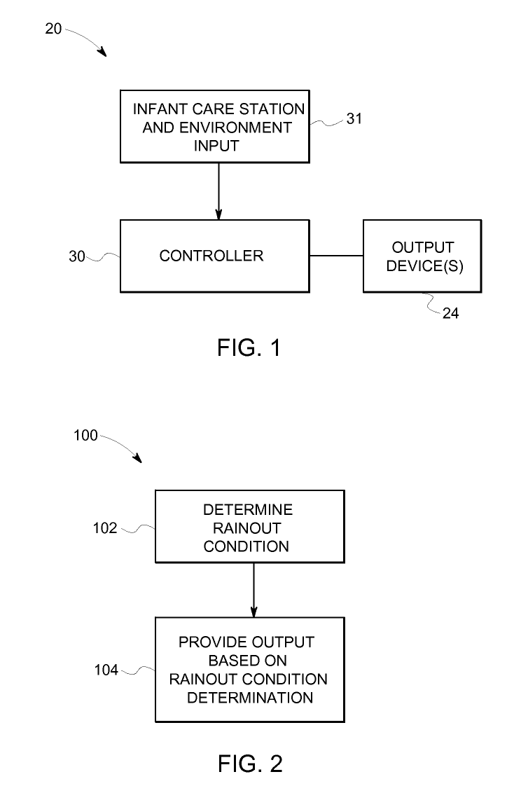

FIG. 1 is a schematic illustration of an example infant care station humidification management system 20. System 20 facilitates healthcare provider management of one or more aspects of humidification of the infant chamber of an infant care station. The infant care station may be in the form of an infant warmer, an incubator or a hybrid warmer/incubator. In the example illustrated in FIG. 1, system 20 determines the occurrence of, or the impending occurrence of a rainout condition, a condition where humidity within infant care station condenses to point that condensation may drip into the infant containing chamber of the infant care station. System 20 further automatically provides output to address the determined or identified rainout condition.

System 20 comprises output device 24 and controller 30. Output device 24 comprises one or more devices that the present information or notifications to a healthcare provider regarding a determined rainout condition and/or automatically adjust infant care station operation based upon a determined rainout condition. In one implementation, output device 24 comprises a display, such as a screen, monitor or the like that presents visible notifications or messages. For example, in one implementation, output device 24 may comprise a monitor that is independent of or built-in as part of an infant care station. In another implementation, output device 24 may comprise the screen of a portable computing or electronic device, such as a personal data assistant, a smart phone, a flash memory player or the like. In another implementation, output device 24 may comprise a wired monitoring station. In implementations where output device 24 comprises a screen or audible device of a portable electronic device, controller 30 is configured to communicate with a portable electronic device in a wireless fashion to cause support electronic device to output a visible and/or audible alarm or message. In one implementation, the output may comprise a visible message. In one implementation, the visible message may additionally or alternatively comprise instructions for the healthcare provider as to how to address the rainout condition.

In another implementation, output device 24 may comprise an audible device. For example, in one implementation output device 24 may comprise an audible alarm. In another implementation, output device 24 may audibly present instructions as to how to respond to a rainout condition. In still other implementations, output device 24 may comprise a light emitting device or alarm that produces a visible light of a particular color, brightness or frequency notifying the healthcare provider of a current or impending rainout condition.

Controller 30 comprises an electronic computing or processing unit configured to determine a current or impending rainout condition and to generate control signals directing one or more output devices 24 to provide output in response to the determined rainout condition. For purposes of this application, the term “processing unit” shall mean a presently developed or future developed processing unit that executes sequences of instructions contained in a memory. Execution of the sequences of instructions causes the processing unit to perform steps such as generating control signals. The instructions may be loaded in a random access memory (RAM) for execution by the processing unit from a read only memory (ROM), a mass storage device, or some other non-transitory persistent storage. In other embodiments, hard wired circuitry may be used in place of or in combination with software instructions to implement the functions described. For example, controller 30 may be embodied as part of one or more application-specific integrated circuits (ASICs). Unless otherwise specifically noted, the controller is not limited to any specific combination of hardware circuitry and software, nor to any particular source for the instructions executed by the processing unit.

In the example illustrated, controller 30 determines a rainout condition in an infant care station based upon temperature gradients between the environment of the infant care station and the interior of the infant chamber of the infant care station. To make such a determination, controller 30 receives input or signals 31 (schematically represented) providing an actual or an estimate of temperatures of the outside environment and the interior infant chamber of the infant care station and humidity of the interior infant chamber of the infant care station. In one implementation, one or more temperature and/or humidity sensors provide one or more signals indicating the internal and/or external temperatures and/or humidities.

In one implementation, a memory associated with controller 30 and storing predefined temperature and humidity settings provides the signals indicating the internal and/or external temperatures and internal humidity. For example, during set up of the infant care station, a healthcare provider may be prompted to enter settings for one or more of the room temperature, the internal infant care station temperature and the internal infant care station humidity, wherein such settings are stored and later retrieved by controller 30 to determine whether a rainout condition, or the probability of a rainout condition (current or impending) exists. In one implementation, the storage settings may additionally include a time component, wherein the settings vary based upon the time of day. In one implementation, both a memory of such stored humidity/temperature settings and humidity/temperature sensors provide signals to controller 30 for use in determining a rainout condition.

FIG. 2 is a flow diagram of an example method 100 that may be carried out by system 20. As indicated by step 102, controller 30 determines a rainout condition. In one implementation, controller 30 receives signals indicating internal and external temperatures with respect to the infant care station as well as internal humidity with respect to the infant care station. Based upon such information, controller 30 determines whether a current rainout condition exists. In yet another implementation, controller 30 receives signals indicating expected changes over time to internal humidity with respect to the infant care station as well as expected changes over time with respect to internal and external temperatures with respect to the infant care station, wherein controller 30 determines an impending or predicted forthcoming rainout condition. In one implementation, controller 30 determines probability of a rainout condition based upon or as a function of a temperature differential across the wall of the incubator cover or hood, the relative humidity level inside the incubator and a scaling constant. In one implementation, the probability of a rainout condition is based on the following example formula:

P(r)=Tw*RH*K1, where:
P(r)=probability of rainout;
Tw=temperature differential across the incubator chamber wall separating the inside and outside of the incubator chamber;
RH=relative humidity inside the incubator chamber; and
K1=a scaling constant.

In one implementation, the temperature differential across the wall may be estimated as the difference between the intern and external (sensed) temperatures, or may be multiplied by an additional scaling constant. In other implementations, controller 30 may determine a present or future rainout condition using other inputs or other formulas.

In some implementations, controller 30 is configured to evaluate and determine or identify a rainout condition during predefined time periods. For example, in one implementation, controller 30 is configured to monitor and/or predict rainout for a user or healthcare provider selected period of time. For instance, a 400 g infant may need a 100% humidity for a seven day period, wherein after the seven-day period, the humidity is subsequently reduced. In such a circumstance, controller 30 may automatically terminate the monitoring for a rainout condition after the predefined seven day period or may automatically terminate monitoring for rainout condition automatically in response to receiving signals indicating that the humidity level within infant care station has been reduced. In yet another implementation, controller 30 may generate control signals causing an alarm or other notification to be output after the seven day period recommending that the healthcare provider reduce the humidity level of the infant care station.

As indicated by step 104, system 20 provides output based upon a current or impending rainout condition determination. In one implementation, controller 30 generates control signals causing output device 24 to provide one or more outputs based upon the determined rainout condition. For example, in one implementation, controller 30 generates control signals causing the output of a visible or audible alarm indicating a current rainout condition or indicating a forthcoming rainout condition. For example, in one implementation, controller 30 may generate control signals causing a wireless notification signal to be transmitted to a portable electronic device serving as output device 24, wherein an alarm or other message is presented on the portable electronic device being carried by an assigned healthcare provider.

In one implementation, some condensation or rainout may be acceptable. In such an implementation, controller 30 generates control signals outputting an alarm in response to a determined amount of rainout exceeding a predetermined non-zero threshold or generates control signals in response to a rainout condition occurring for a predetermined non-zero minimum period of time.

In yet another implementation, controller 30 generates control signals causing output device 24 to present a visible or audible message providing a suggestion or recommendation for a healthcare provider to take specific actions to address the current or forthcoming rainout condition. For example, controller 30 may instruct the healthcare provider to increase the temperature or lower the humidity within the infant care station to stop or reduce rainout within the infant care station or to delay the time at which a rainout condition is triggered. Controller 30 may instruct the healthcare provider to perform cleaning at a specific time following the identified rainout condition or to perform more frequent cleaning of the infant care station in response to a determined rainout condition.

In one implementation, controller 30 may alternatively or additionally generate control signals outputting a message, alarm or other notification in response to a predetermined period of time following the time at which a rainout condition is determined to initially exist or based upon a determined extent of rainout. In one implementation, controller 30 may utilize the determined rainout condition, such as the time that a rainout condition begins or the extent of such rainout, in combination with other factors such as temperature, ventilation, infant characteristics and the like to determine a recommended time for cleaning the infant care station to reduce infection risks. Based upon the determined recommended time for cleaning the infant care station, controller 30 may generate control signals causing a cleaning alarm or cleaning notification to be output. For example, in one implementation, controller 30 may automatically transmit a cleaning notification or reminder to a display screen of the infant care station, monitoring station and/or to a portable electronic device, such as a personal date assistant, smart phone or the like, of an assigned healthcare provider.

In yet another implementation, controller 30 generates control signals that automatically adjust operation of the infant care station based upon the determined rainout condition (present or future). For example, in response to a determined present rainout condition, controller 30 may generate control signals automatically adjusting the internal temperature of the infant care station or the internal humidification level of the infant care station.

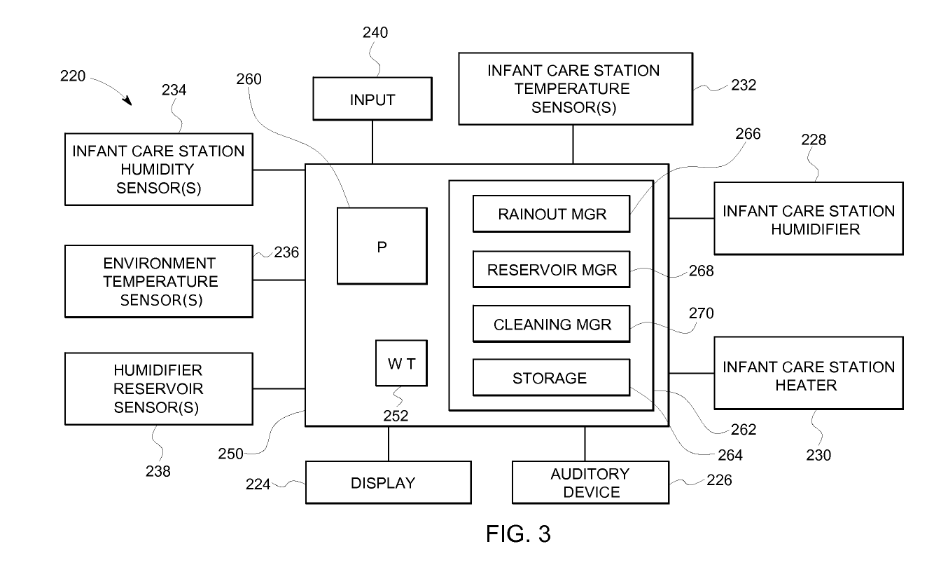

FIG. 3 schematically illustrates an example infant care station humidification management system 220. As with system 20, system 220 facilitates healthcare provider management of one or more aspects of humidification of the infant chamber of an infant care station. In the example illustrated in FIG. 3, system 220 manages humidification and rainout, manages the level of water or other liquid within the humidifier reservoir and manages cleaning of the infant care station based in part upon humidification and rainout.

System 20 comprises output devices: display 224, auditory device 226, infant care station humidifier 228 and infant care station heater 230; input devices: infant care station temperature sensors 232, infant care station humidity sensors 234, environment temperature and humidity sensors 236, humidifier reservoir sensors 238, and input 240; and controller 250. Display 224 comprises a screen, monitor or the like that presents visible notifications or messages. For example, in one implementation, display 224 may comprise a monitor independent of or built-in as part of an infant care station. In another implementation, display 224 may comprise the screen of a portable computing or electronic device, such as a personal data assistant, a smart phone, a flash memory player or the like. In implementations where display 224 comprises a screen or audible device of a portable electronic device, controller 30 is associated with a wireless transceiver 252 to communicate with the portable electronic device in a wireless fashion to cause the portable electronic device to output a visible and/or audible alarm or message. In still other implementations, display 224 may comprise a light emitting device or alarm that produces a visible light of a particular color, brightness or frequency notifying the healthcare provider of a current or impending rainout condition. In one implementation, the output may comprise a visible message. In one implementation, the visible message may additionally or alternatively comprise instructions for the healthcare provider as to how to address the rainout condition.

Auditory device 226 comprises a device to output audible sounds, words or sentences. For example, in one implementation auditory device may comprise a speaker to produce an audible alarm. In another implementation, auditory device 226 is configured to audibly present instructions as to how to respond to a rainout condition. In some implementations, one or both of display 224 and auditory device 226 may be omitted.

Infant care station humidifier 228 comprises a device to evaporate an evaporant, such as water, to increase relative humidity of air within the infant chamber of an infant care station. In one implementation, humidifier 228 comprises an evaporative humidifier in which air is passed through a screen, wick or other evaporate carrier to pick up moisture. In yet another implementation, humidifier 228 comprises a steam humidifier or vaporizer in which water is boiled. In yet another implementation, humidifier 228 may comprise an impeller in which a rotating disk flings water at a comb-like diffuser to break the water into droplets that float in the air. In one implementation, humidifier 228 is adjustable such that the rate at which water or water vapor is added to increase humidity may be changed.

Infant care station heater 230 comprises a device which heats the air within the infant chamber to a predefined temperature or within a predefined temperature range. In one implementation of heater 230 comprises a radiant heating or warming device. In other implementations, heater 230 may comprise other types of heating and warming devices, such as a convective or conductive heating devices.

Infant care station temperature sensors 232 and infant care station humidity sensors 234 sense and detect the temperature and humidity, respectively, within the infant chamber (below the hood). In one implementation, sensors 232, 234 are in the form of probes positioned adjacent to the region within the infant care station in which an infant rests. In one implementation, the infant care station may be configured to co-bed multiple infants, wherein one or more probes are provided for each of the multiple infants within the infant care station. In one implementation, successors may be supported by a structure of the infant care station. In another implementation, such sensors may be provided on sensing structures that attach to the skin of the infant. In some implementations, one or both of such sensors 232, 234 may be omitted such as where stored temperature and/or humidity setting values are utilized for determining rainout conditions.

Environment temperature and humidity sensors comprise sensors configured to sense the temperature outside of the infant care station, such as adjacent an external to the canopy or hood of the infant care station. In some implementations, sensor 236 may be omitted, where stored external or environmental temperature and/or internal humidity setting values are utilized for determining rainout conditions within the infant care station.

Humidifier reservoir sensors 238 comprise sensors to detect the level of evaporant or water within a humidifier reservoir of an infant care station. In other implementations, such sensor may be omitted where the level of evaporant or water within the humidifier reservoir is estimated by controller 250 based upon an initial fill volume and an estimated rate at which water is consumed by the humidifier. In yet other implementations, such sensors 238 may be omitted.

Input 240 comprises one or more devices by which a healthcare provider may input commands, selections, data or settings for system 220 and the associated infant care station. In one implementation, input 240 may comprise a keyboard, touchpad, touch screen, mouse, microphone with speech recognition software and the like. Input 240 facilitates input of internal infant care station temperatures, external or environmental temperatures, internal infant care station humidities as well as other information to facilitate the determination of a rainout condition by controller 250. Input 240 further facilitates input of other settings such as a healthcare provider determined time period during which controller 250 is to monitor rainout conditions, infant characteristics, alarm overrides, desired humidity settings (humidity level and time), infant care station temperatures, and notification or alarm settings such as alarm thresholds, alarm characteristics, healthcare provider assignments and communication addresses to which alarms or messages are to be transmitted.

Controller 250 assists in the management of humidification of the associated infant care station. In one implementation, controller 250 comprises an electronic device independent of the infant care station, wherein sensors 232, 234 may be mounted or otherwise secured with respect to the infant care station. In such an implementation, controller 250 may be added to and existing infant care station. In one implementation, controller 250 or portions of controller 250 may be remotely located relative to the associated infant care station. For example, in one implementation, portions of controller 250 may be located across a local area network or a wide area network, such as in the “cloud” across the Internet, wherein controller 250 communicate with the associated infant care station across the wired or wireless network. In one implementation, controller 250 may be provided by a regional computing center or may be provided by a manufacturer or distributor of the infant care station. In yet other implementation, controller 250 may be incorporated as part of the controller or electronic device of the infant care station itself.

Controller 250 comprises processing unit 260 and memory 262. Processing unit 260 comprises one or more processing units configured to carry out instructions or code contained in memory 262. Memory 262 comprises a non-transitory computer-readable medium, such as a flash memory, optical memory disk, magnetic memory disk or the like. Memory 262 comprises storage 264, rainout manager module 266, reservoir manager module 268 and cleaning manager module 270. In one implementation, processor 260 and memory 262 are locally located with respect to the associated infant care station. In one implementation, processor 260 and memory 262 are located remote with respect to the associated infant care station.

Storage 264 comprises that portion memory 262 that stores data and other information pertaining to system 220 and the associated infant care station. Storage 264 stores healthcare provider input internal infant care station temperatures, external or environmental temperatures, internal infant care station humidities as well as other information to facilitate the determination of a rainout condition by controller 250. Storage 264 further stores input settings such as a healthcare provider determined time period during which controller 250 is to monitor rainout conditions, infant characteristics, alarm overrides, desired humidity settings (humidity level and time), infant care station temperatures, and notification or alarm settings such as alarm thresholds, alarm characteristics, healthcare provider assignments and communication addresses to which alarms and notifications are to be sent.

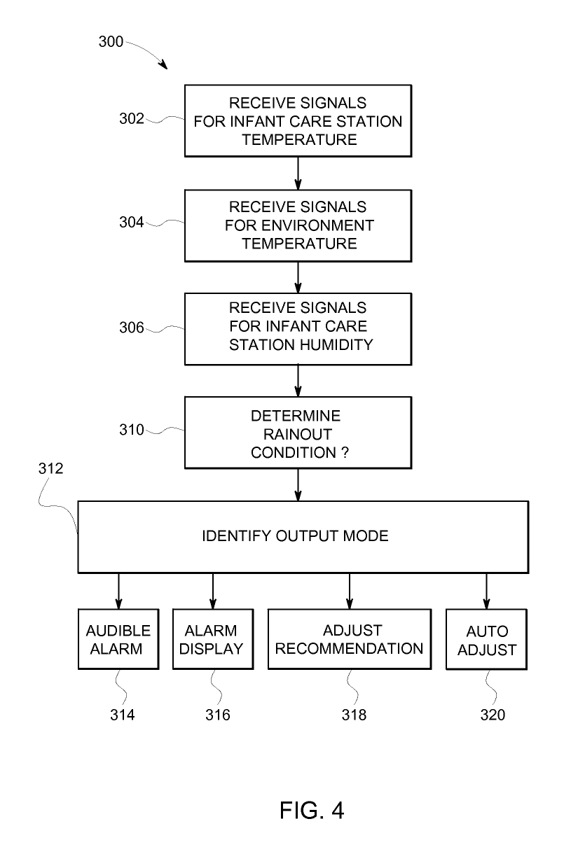

Rainout manager module 266 comprises computer-readable instructions for directing processor 260 to identify an existing or impending rainout condition and to generate control signals in response to the determined rainout condition. In one implementation, rainout manager module 266 comprises computer-readable instructions or code configured to direct processing unit 260 to carry out method 100 described above with respect to FIG. 2. In another implementation, rainout manager module 266 comprises computer-readable instructions or code configured to carry out method 300 illustrated in FIG. 4. As indicated by steps 302, 304 of method 300 illustrated in FIG. 4, rainout manager module 266 directs processing unit 260 to retrieve or otherwise receive signals indicating the existing and/or upcoming internal temperature of an infant chamber of the infant care station and signals indicating the existing or expected external environmental temperature about the infant care station. In some implementations, such signals may be received from one or more temperature sensors. In some implementations, such signals may be received or retrieved from storage 264 or may be estimated or calculated from other data maintained in storage 264.

As indicated by step 306 of method 100 illustrated in FIG. 4, rainout manager module 266 further directs processing unit 260 to retrieve or otherwise receive signals indicating the existing and/or upcoming internal humidity level of an infant chamber of the infant care station. In some implementations, such signals may be received from one or more humidity sensors. In some implementations, such signals may be received or retrieved from storage 264 or may be estimated or calculated from other data maintained in storage 264.

As indicated by step 310, rainout manager module 266 directs processing unit 260 to utilize the temperatures for the interior of the infant care station and the environment and the humidity for the interior of the infant care station to calculate or determine whether a rainout condition currently exists or to estimate when a rainout condition is expected to occur. In one implementation, controller 30 determines a probability of a rainout condition based upon or as a function of a temperature differential across the wall of the incubator cover or hood, the relative humidity level inside the incubator and a scaling constant. In one implementation, the probability of a rainout condition is based on the following example formula:

P(r)=Tw*RH*K1, where:
P(r)=probability of rainout;
Tw=temperature differential across the incubator chamber wall separating the inside and outside of the incubator chamber;
RH=relative humidity inside the incubator chamber; and
K1=a scaling constant.

In one implementation, the temperature differential across the wall may be estimated as the difference between the intern and external (sensed) temperatures, or may be multiplied by an additional scaling constant. In other implementations, controller 30 may determine a present or future rainout condition using other inputs or other formulas.

As indicated by step 312, rainout manager module 266 directs processor or processing unit 260 to identify which output mode or modes have been selected by the healthcare provider. In one implementation, such settings are stored in storage 264. Based upon such previously stored user selections, rainout manager module 266 directs processing unit 260 to output one or more of various selectable outputs: an audible alarm per step 314; a visible alarm per step 316 such as an alarm on a display screen or an alarm by a light emitting device; a visible or audible recommendation for an adjustment of the operation of the infant care station by the healthcare provider per step 318; or the output of control signals that automatically adjust operation of the infant care station per step 320. Such adjustment of the infant care station, whether recommended per step 318 or automatically adjusted per step 320, may comprise adjustment of the rate at which humidity is added to the infant chamber of the infant care station by infant care station humidifier 228 or may comprise an adjustment of the temperature of the interior of the infant care station by infant care station heater 230.

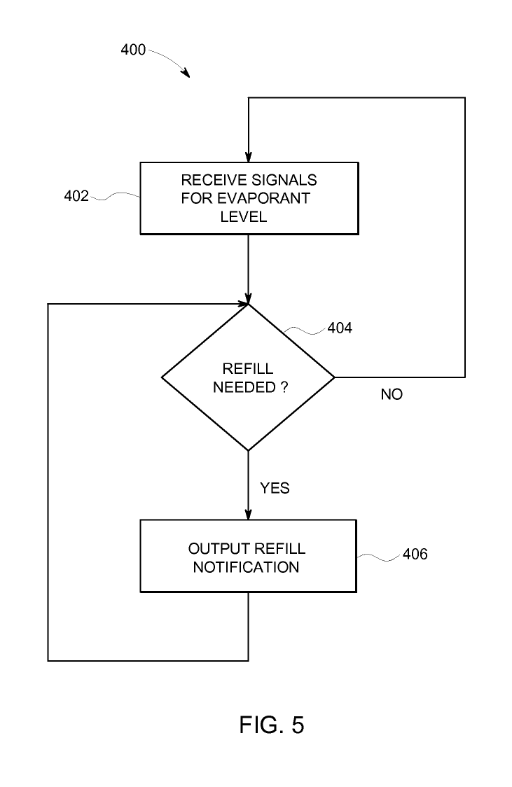

Reservoir manager module 268 comprises computer-readable instructions to direct processing unit 260 to manage the amount or level of evaporant or water within the reservoir of the humidifier of the infant care station. Module 268 provides instructions for processing unit 260 to notify a healthcare provider of when additional evaporative water should be added to the humidifier reservoir of the infant care station. In one implementation, module 268 directs processing unit 260 to carry out method 400 shown in FIG. 5. As indicated by step 402 of method 400 illustrated in FIG. 5, reservoir manager module 268 direct processing unit 260 to retrieve or otherwise receive signals indicating the level of a evaporative or water within the humidifier level. In one implementation, processing unit 260 receive such signals from sensors 238. In yet another implementation, processing unit 260 may receive such signals from a level estimator module which estimates level of evaporant or water based upon initial reservoir fill volume and an estimated rate at which evaporant has been consumed.

As indicated by step 404, module 268 directs processing unit 260 to determine whether a refill of the humidifier reservoir is needed. In one implementation, the determination of whether a refill of the humidifier reservoir is needed is made by comparing the determined current level with a predefined fill threshold stored in storage 264. In another implementation, the determination of whether a refill of the humidifier reservoir is needed is made based upon the current level and the estimated rate at which the evaporant is expected to be consumed. As indicated by step 406, if processing unit 260 determines that a refill is needed, module 268 directs processing unit 260 to generate control signals notifying a healthcare provider that the reservoir should be filled with additional evaporant. In one implementation, such notification may be provided by one or more audible or visible alarms. In yet another implementation, such notification may be divided by an audible or visible message providing additional information such as when the reservoir is expected to become empty at the existing humidity level. In some implementations, reservoir manager module 268 may be omitted.

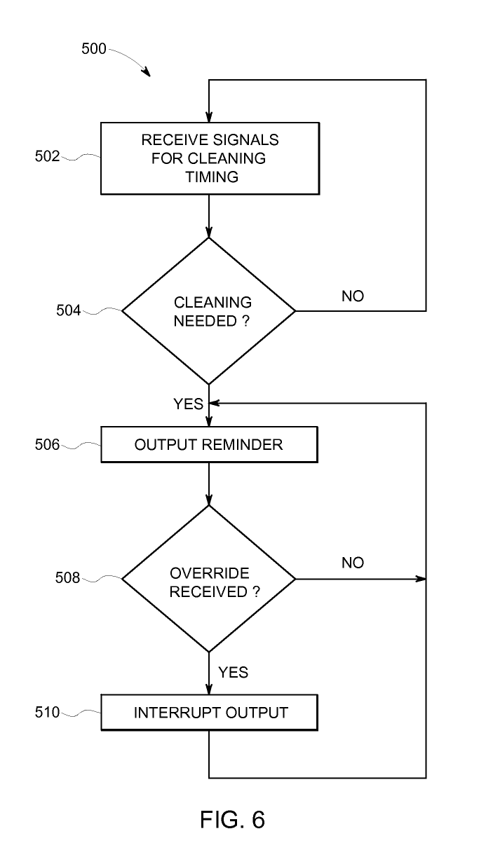

Cleaning manager module 270 comprises a computer-readable medium containing instructions or code for directing processing unit 260 to assist in the management of the cleaning or cleanliness of the infant care station. In the example illustrated, cleaning manager module 270 utilizes the determined rainout conditions as a factor in the management of the cleaning of the infant care station. In one implementation, cleaning manager module 270 is configured to carry out method 500 illustrated in FIG. 6.

As indicated by step 502 of method 500 and FIG. 6, module 270 instructs or directs processing unit 260 to retrieve or otherwise receive signals indicating timing for cleaning of the infant care station. In one implementation, such signals are based upon predefined for previously stored timing intervals in storage 264. In one implementation, such timing intervals are based upon manufacturer or healthcare industry recommended cleaning intervals. In one implementation, module 270 may direct processing unit 2602 prompt a healthcare provider to input, by input 240, intervals for the cleaning of the infant care station.

In one implementation, module 270 may further direct processing unit 260 to establish new intervals or adjust previously stored or set cleaning intervals based upon previously determined rainout conditions. In one implementation, module 270 may direct processing unit 260 to reduce the recommended cleaning intervals in circumstances where processing unit 260 has determined or identified a rainout condition continuing for a period of time or for collective periods of time since the last cleaning of the infant care station that exceed a predefined threshold. In one implementation, module 270 may direct processing unit 260 to reduce the recommended cleaning intervals in circumstances where processing unit 260 has determined or identified and extent or degree of rainout condition (the estimated amount of rainout) since the last cleaning of the infant care station that exceeds a predefined threshold. In such circumstances, cleaning manager module 270 adapts to or takes into account the impact of determined rainout conditions on the frequency at which the infant care station or portions of the infant care station should be cleaned to reduce contamination or infection concerns.

As indicated by step 504, module 270 determines whether cleaning of the infant chamber of the infant care station is needed and/or determines a future time at which cleaning of the infant chamber the infant care station is needed. As indicated by step 506, at the time that cleaning is needed, cleaning manager 270 directs processing unit 260 to output a cleaning reminder. The output may be provided in the form of an audible or visible alarm or an audible or visible message.

As indicated by step 508, in the example illustrated, module 270 further provides the healthcare provider with an opportunity to override the notice or alarm. For example, in certain circumstances, it may not be possible to remove an infant or infants from the infant chamber of the infant care station to clean infant care station to allow cleaning In such a circumstance, system 220 allows a healthcare provider to input an override through input 240. As indicated by step 510, in response to receiving input indicating an override of the notice or reminder, the alarm, message or other output is temporarily interrupted for a predetermined period of time. In one implementation, the time period during which the alarm or notice is interrupted may be input by the healthcare provider through input 240 and stored in memory 264. Once the time period for interrupting the notice or alarm has expired, module 270 directs processing unit 260 to once again output a reminder per step 506.

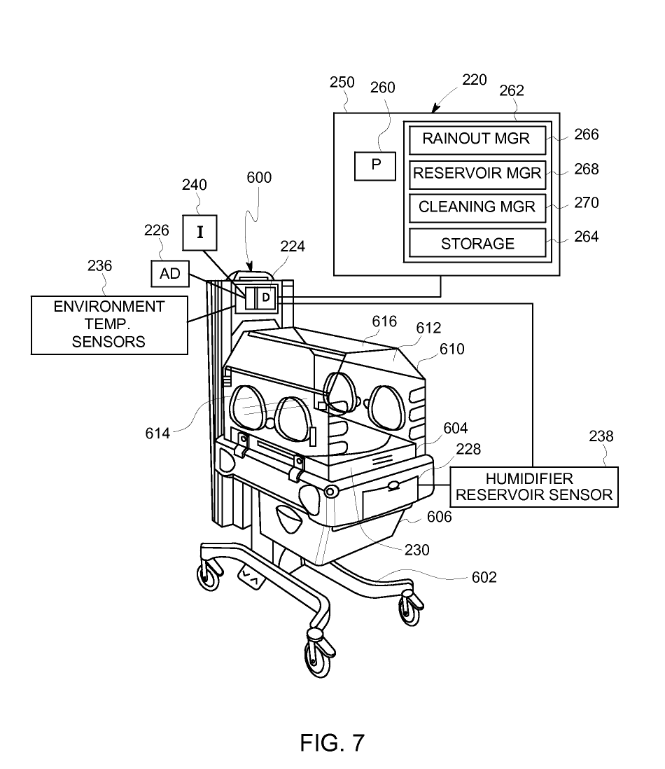

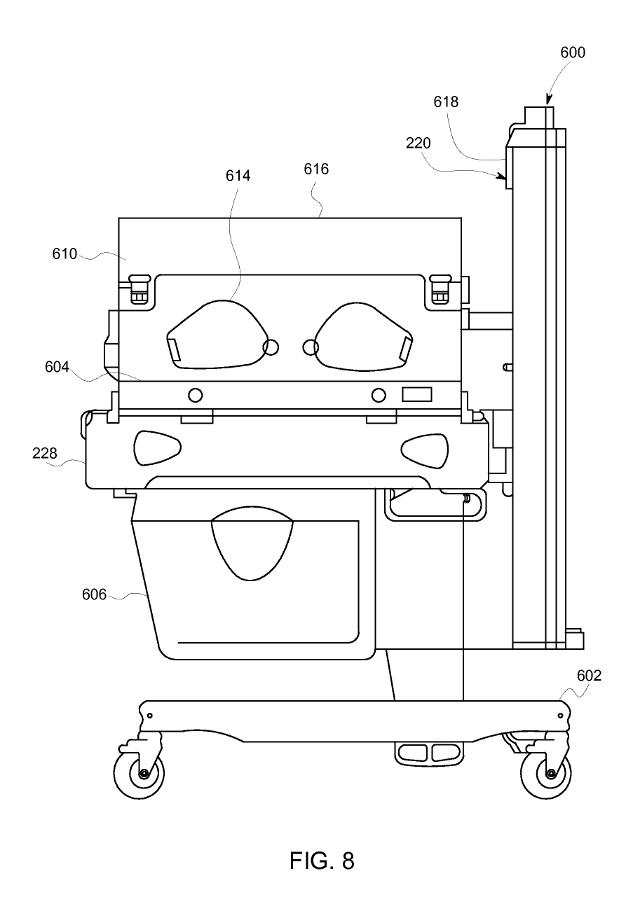

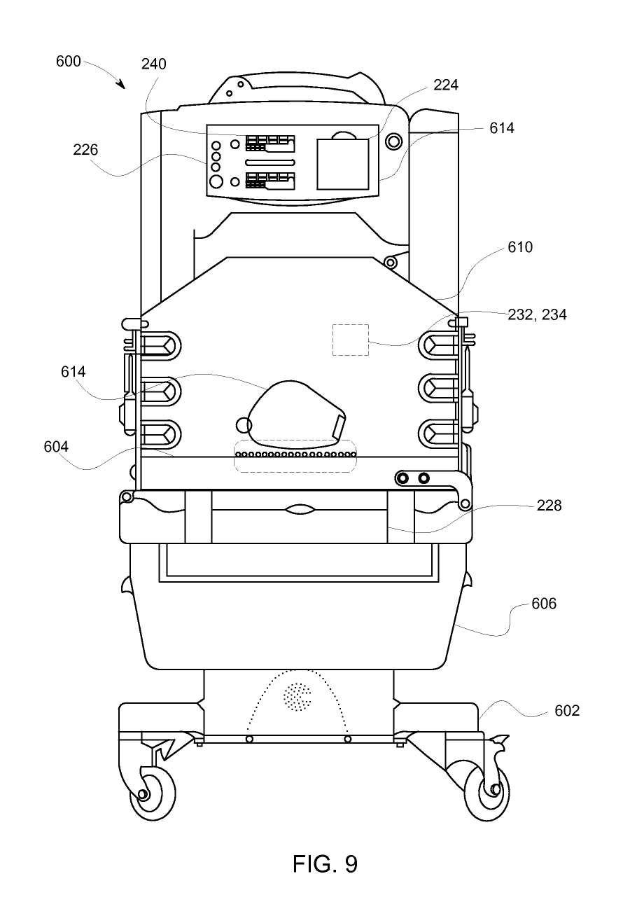

FIGS. 7-9 illustrate an example infant care station 600 comprising infant care station humidification management system 220. Infant care station 600 is configured to provide a regulated or controlled neonatal infant microenvironment for one or more neonates or infants. Although infant care station 600 is illustrated as physically incorporating system 220 as part of its electronics, in other implementations, infant care station 600 is independent of system 220 (but for the provision of humidifier 228, heater 230 and temperature/humidity sensors carried by infant care station 600), wherein infant care station 600 communicates with controller 220 across a wired or wireless network.

In the example illustrated, in addition to system 220, infant care station 600 comprises base 602, bed 604, humidifier reservoir 606, hood 610, and control panel 614 providing display 224, auditory device 226 and input 240. Base 602 supports infant care station 600 at a selected height and facilitates transit of infant care station 600.

Bed 604 comprises a mattress or other structure upon which a neonatal infant may rest. In one implementation, bed 604 incorporates radiant infant care station heating components 230. In another implementation, radiant heating components 230 may provide along a side of the chamber provided by hood 610.

Humidifier reservoir 606 comprises a tank or other volume containing evaporant or water for infant care station humidifier 228. Although illustrated as being located below bed 230, reservoir 606 may be provided other locations.

Hood 610 comprises a transparent housing extending about bed 604 to form a chamber 612 providing the microenvironment for one or more infants. In the example illustrated, hood 610 includes a plurality of portholes 614 through which a healthcare provider may access the one or more infants within the chamber 612. In the example illustrated, hood 610 comprises a single structure movable as a unit. In other implementations, sidewalls of hood 610 may remain about bed 604 forming a crib, wherein the topmost portion or canopy 616 is separable from the sidewalls of hood 610 to access one or more infants.

Control panel 614 facilitates the display and input of information with regard to infant care station 600. Control panel 614 comprises display 224, auditory device 226 and input 240 of system 220. As noted above, in some implementations, display 224, auditory device 226 and input 240 may be additionally or alternatively provided by other electronic devices distinct from station 600 such as an independent monitoring console or a portable electronic device to be carried by a physician or healthcare provider.

Although the present disclosure has been described with reference to example embodiments, workers skilled in the art will recognize that changes may be made in form and detail without departing from the spirit and scope of the claimed subject matter. For example, although different example embodiments may have been described as including one or more features providing one or more benefits, it is contemplated that the described features may be interchanged with one another or alternatively be combined with one another in the described example embodiments or in other alternative embodiments. Because the technology of the present disclosure is relatively complex, not all changes in the technology are foreseeable. The present disclosure described with reference to the example embodiments and set forth in the following claims is manifestly intended to be as broad as possible. For example, unless specifically otherwise noted, the claims reciting a single particular element also encompass a plurality of such particular elements.

What is claimed is:

1. An infant care station humidification management system comprising:
an output device; and
a controller to generate control signals based upon a determined rainout condition, wherein the output device provides output in response to the control signals.

2. The infant care station humidification management system of claim 1, wherein the output comprises an alert.

3. The infant care station humidification management system of claim 2, wherein the output comprises a visible message.

4. The infant care station humidification management system of claim 1, wherein the output comprises a visible message comprising instructions to address the rainout condition.

5. The infant care station humidification management system of claim 1, wherein the output comprises control signals automatically adjusting operation of the infant care station.

6. The infant care station humidification management system of claim 1, wherein the controller is configured to prompt for input regarding a desired humidity level within the infant care station.

7. The infant care station humidification management system of claim 1, wherein the controller is configured to receive input regarding an internal temperature of the infant care station and wherein the rainout condition is determined based on the input.

8. The infant care station humidification management system of claim 1, wherein the controller is configured to receive input regarding an external temperature adjacent the infant care station and wherein the rainout condition is determined based on the input.

9. The infant care station humidification management system claim 1, wherein the controller is configured to receive a sensed internal temperature of the infant care station and wherein the rainout condition is determined based on the sensed internal temperature.

10. The infant care station humidification management system of claim 1, wherein the controller is configured to receive sensed external temperature adjacent to the infant care station and wherein the rainout condition is determined based on the sensed external temperature.

11. The infant care station humidification management system of claim 1, wherein the output comprises an automatic adjustment of at least one of an internal temperature or humidity of the infant care station.

12. The infant care station humidification management system of claim 1, wherein the controller is configured to monitor the determined rainout condition for a care person selected time.

13. The infant care station humidification management system of claim 1, wherein the controller is configured to generate second control signals based upon a determined water reservoir level of a humidifier of the infant care station and water consumption by the humidifier, wherein the output device provides the output in response to the second control signals.

14. The infant care station humidification management system of claim 13, wherein the controller is configured to generate second control signals at predetermined time intervals and wherein the output device is configured to request humidifier operation verification in response to the second control signals.

15. The infant care station humidification management system of claim 13, wherein the controller is configured to generate second control signals at time intervals based upon operation of a humidifier of the infant care station, wherein the output device is configured to present infant care station cleaning reminders in response to the second control signals.

16. The infant care station humidification management system of claim 15, wherein the predetermined time intervals are based upon the determined rainout condition.

17. The infant care station humidification management system of claim 1, wherein the controller is configured to monitor and determine a rainout condition for a predetermined period of time based upon input by a healthcare provider.

18. A method comprising:
determining a rainout condition with respect to an infant care station; and
providing an output based upon the determined rainout condition.

19. The method of claim 18, wherein the output comprises an alarm.

20. The method claim 18, wherein the output comprises a visible message comprising instructions to address the rainout condition.

21. The method of claim 18, further comprising prompting for input regarding a desired humidity level within the infant care station.

22. The method of claim 18, wherein the controller is configured to receive input regarding at least one of an internal temperature of the infant care station and an external temperature adjacent to the infant care station and wherein the rainout condition is determined based on at least one of the internal temperature of the infant care station and the external temperature adjacent to the infant care station.

23. The method claim 18, further comprising receiving at least one of a sensed internal temperature of the infant care station and a sensed external temperature adjacent to the infant care station, wherein the rainout condition is determined based on at least one of the sensed internal temperature and the sense external temperature.

24. The method claim 18, wherein the output comprises an automatic adjustment of at least one of an internal temperature or humidity of the infant care station.

25. The method claim 18, wherein the output displays a calculated value for at least one proposed change to an internal temperature or humidity of the infant care station

26. An apparatus comprising:
a non-transitory computer-readable medium containing computer-readable code to direct a processor to:
determine a rainout condition with respect to an infant care station; and
generate control signals to provide an output based upon the determined rainout condition.

27. The apparatus of claim 26, wherein the output comprises an alarm.

28. The apparatus claim 26, wherein the output comprises a visible message comprising instructions to address the rainout condition.
